(19) United States
(12) Patent Application Publication
(10) Pub. No.: US 2016/0354427 A1
(43) Pub. Date: Dec. 8, 2016

(54) METHODS FOR INDUCING EPITHELIAL CANCER CELL SENESCENCE

(71) Applicant: Institute for Cancer Research d/b/a The Research Institute of Fox Chase Cancer Center, Philadelphia, PA (US)

(72) Inventors: Rugang Zhang, Elkins Park, PA (US); Benjamin Bitler, Cheltenham, PA (US)

(21) Appl. No.: 15/244,083

(22) Filed: Aug. 23, 2016

Related U.S. Application Data
(62) Division of application No. 13/963,031, filed on Aug. 9, 2013.
Application No. 13/963,031 is a continuation of application No. PCT/US2012/024648, filed on Feb. 10, 2012.
(60) Provisional application No. 61/441,409, filed on Feb. 10, 2011.
(60) Provisional application No. 61/445,145, filed on Feb. 22, 2011.

(51) Int. Cl. A61K 38/08; G01N 33/574; C12Q 1/68

(52) U.S. Cl. CPC A61K 38/08; C12Q 1/6886; G01N 33/57449; C12Q 2600/158; C12Q 2600/106; G01N 2800/52

(57) ABSTRACT

Systems, methods, and computer readable media for diagnosing or characterizing epithelial cancer or its stages based on the level of expression of the Wnt5a gene or protein are provided. The level of nucleic acids encoding Wnt5a or the level of Wnt5a protein is measured in a tissue sample, and the level is compared with reference values. Methods for inducing senescence of an epithelial cancer cell are also provided.

### CROSS REFERENCE TO RELATED APPLICATIONS

This application is a divisional of U.S. patent application Ser. No. 13/963,031 filed on Aug. 9, 2013 which is a continuation of PCT Application No. PCT/US2012/024648 filed on Feb. 10, 2012, and claims priority to U.S. Provisional Application No. 61/441,409 filed on Feb. 10, 2011 and U.S. Provisional Application No. 61/445,145 filed on Feb. 22, 2011. The contents of each application are incorporated by reference herein, in their entirety and for all purposes.

### REFERENCE TO A SEQUENCE LISTING

This application includes a Sequence Listing submitted electronically as a text file named Wnt5a Sequence Listing_St25, created on Aug. 9, 2013, with a size of 12,000 bytes. The Sequence Listing is incorporated by reference herein.

### FIELD OF THE INVENTION

The invention relates generally to the fields of cancer biology and personalized medicine. More particularly, the invention relates to methods for promoting senescence in epithelial cancer cells by enhancing Wnt5a levels, and to methods for diagnosing early or late stage epithelial cancers based on determinations of Wnt5a levels in patients.

### BACKGROUND OF THE INVENTION

Various publications, including patents, published applications, technical articles and scholarly articles are cited throughout the specification. Each of these cited publications is incorporated by reference herein, in its entirety and for all purposes.

The human Wnt gene family consists of 19 members, encoding evolutionarily conserved glycoproteins. Wnt signaling is initiated by binding of a Wnt ligand to its cognate Frizzled receptor, and canonical Wnt signaling results in stabilization of the key transcription factor β-catenin, which then translocates into the nucleus and drives the expression of proliferation-promoting genes. In mammals, canonical Wnt signaling maintains the proliferation potential of many tissue stem/progenitor cells, including hematopoietic cells and the intestinal epithelium. Consistent with its proliferation-promoting function, canonical Wnt signaling is often activated in human cancer cells.

Wnt signaling is required for proper ovary development and function. In a murine ovarian cancer model, activation of canonical Wnt signaling cooperates with inactivation of the tumor suppressor PTEN in driving ovarian carcinogenesis. The role of Wnt signaling in ovarian cancer, however, remains poorly understood.

Downregulation of canonical Wnt signaling has previously been shown to induce cell senescence by activating the histone repressor A (HIRA)/promyelocytic leukemia (PML) pathway. Consistent with its senescence-promoting function, inactivation of PML suppresses senescence. Cellular senescence is recognized as a potent tumor suppressor mechanism. Indefinite cell cycle arrest in transformed cells inhibits tumor growth and prevents further progression. Understanding mechanisms that promote senescence could lead to new clinical approaches in the treatment of cancer. Hence, driving cancer cells to undergo senescence represents an avenue for cancer therapeutics.

### SUMMARY OF THE INVENTION

The invention features methods for diagnosing or characterizing the stage of an epithelial cancer in a subject such as a human being. In some aspects, the methods comprise determining the level of expression of a nucleic acid encoding Wnt5a in a tissue sample obtained from a subject, comparing the determined level of expression with one or more reference values for the expression of the nucleic acid encoding Wnt5a in the tissue, using a processor programmed to compare determined levels and reference values, and characterizing the stage of the epithelial cancer based on the comparison. In some aspects, the methods comprise determining the concentration of Wnt5a protein in a tissue sample obtained from a subject, comparing the determined concentration with one or more reference values for the concentration of Wnt5a protein in the tissue, using a processor programmed to compare the determined concentration and reference values, and characterizing the stage of the epithelial cancer based on the comparison.

The methods may be used for any epithelial cancer, non-limiting examples of which include an epithelial cancer of the ovary, colon, rectum, breast, prostate, pancreas, esophagus, bladder, liver, uterus, or brain. The reference values may comprise one or more of Wnt5a nucleic acid or protein expression levels associated with stage I epithelial cancer, expression levels associated with stage II epithelial cancer, expression levels associated with stage III epithelial cancer, expression levels associated with stage IV epithelial cancer, or expression levels in a healthy subject.

The methods may further comprise determining the prognosis of the subject based on the comparison. The methods may further comprise treating the subject with a regimen capable of improving the prognosis of a patient having an epithelial cancer. The treatment regimen may comprise enhancing the expression of the Wnt5a gene in the subject. The treatment regimen may comprise downregulating aspects of a cell's biochemistry that cause a reduced expression of Wnt5a. The treatment regimen may comprise administering to the subject a therapeutically effective amount of the Wnt5a protein, or biologically active fragment thereof.

The invention also features systems for diagnosing or characterizing the stage of an epithelial cancer. The systems may comprise a data structure comprising one or more reference values comprising one or more of Wnt5a nucleic acid or Wnt5a protein expression levels associated with stage I epithelial cancer, expression levels associated with stage II epithelial cancer, expression levels associated with stage III epithelial cancer, expression levels associated with stage IV epithelial cancer, or expression levels in a healthy subject, and a processor operably connected to the data structure. The processor may be programmed to compare the level of expression of Wnt5a nucleic acids or proteins determined from a tissue sample obtained from a subject with the reference values. The processor may be a computer processor. The system may further comprise a computer network connection.

The invention also features computer readable media. The computer readable media may comprise executable code for causing a programmable processor to compare the expression level of a nucleic acid encoding Wnt5a, or the Wnt5a protein, in a tissue sample obtained from a subject with one or more reference values comprising one or more of Wnt5a nucleic acid or Wnt5a protein expression levels associated with stage I epithelial cancer, expression levels associated with stage II epithelial cancer, expression levels associated with stage III epithelial cancer, expression levels associated with stage IV epithelial cancer, or expression levels in a healthy subject. The computer readable media may optionally comprise executable code for causing a programmable processor to determine a prognosis for an epithelial cancer patient from a comparison of the determined level of expression of the Wnt5a nucleic acid or Wnt5a protein with the reference values. The computer readable media may optionally comprise executable code for causing a programmable processor to recommend a treatment regimen for treating an epithelial cancer patient.

The invention also features methods for inducing senescence in an epithelial cancer cell. In general, the methods comprise enhancing the level of expression of the Wnt5a gene in an epithelial cancer cell. The epithelial cancer cell may be an epithelial cancer cell of the ovary, colon, rectum, breast, prostate, pancreas, esophagus, bladder, liver, uterus, or brain. Enhancing the level of expression of the Wnt5a gene preferably activates the HIRA pathway in the cell, and/or antagonizes the Wnt canonical signal pathway in the cell, and/or antagonizes beta-catenin signaling in the cell. In alternative aspects, the methods may comprise contacting an epithelial cancer cell with an amount of Wnt5a protein, or biologically active fragment thereof, effective to induce senescence in the cell.

### BRIEF DESCRIPTION OF THE DRAWINGS

FIGS. 1A-1F show that Wnt5a is expressed at significantly lower levels in human EOC cells compared with normal human ovarian surface or fallopian tube epithelial cells, and a lower level of Wnt5a expression predicts shorter overall survival in human EOC patients. FIG. 1A shows expression of Wnt5a mRNA in primary HOSE cells and the indicated human EOC cell lines was determined by semiquantitative RT-PCR. Expression of glyceraldehyde 3-phosphate dehydrogenase (GAPDH) mRNA was used as a loading control. FIG. 1B shows Wnt5a mRNA levels that were quantified by qRT-PCR in 6 individual isolations of primary HOSE cells and 7 different EOC cell lines. Expression of β-2-microglobulin was used to normalize Wnt5a mRNA expression. *, P=0.008 compared with human EOC cells. FIG. 1C is the same as FIG. 1A, but FIG. 1C examined for Wnt5a and GAPDH protein expression by immunoblotting. FIG. 1D shows examples of Wnt5a IHC staining in normal human ovarian surface epithelium, fallopian tube epithelium, and EOC of indicated histologic subtypes. Bar, 50 μm. Arrows point to examples of positively stained human ovarian surface epithelial cells and fallopian tube epithelial cells. FIG. 1E shows representative images from tissue microarray depicting low Wnt5a expression correlated with high Ki-67, a cell proliferation marker. FIG. 1F shows loss of Wnt5a expression is an independent poor prognosis marker in human EOC patients. A lower level of Wnt5a expression correlates with shorter overall survival in human EOC patients. The univariate overall survival curve (Kaplan-Meier method) for EOC patients (n=123) with high- or low-Wnt5a expression as determined by immunohistochemical analysis.

FIGS. 2A-2C show promoter DNA CpG island hypermethylation contributes to Wnt5a downregulation in human EOC cells. FIG. 2A shows schematic structure of the human Wnt5a gene transcript and its promoter CpG islands. Locations of exon 1 (open rectangle), CpG sites (vertical lines) and coding exons (filled rectangle), and the transcription start site (curved arrow) are indicated. Flat arrows indicate the positions of primers used for PCR amplification, and the size of PCR product is also indicated. FIG. 2B shows PEO1 cells that were treated with 5 μmol/L Aza-C for 4 days, and mRNA was isolated from control- and Aza-C-treated cells and examined for Wnt5a mRNA expression by qRT-PCR. Mean of 3 independent experiments with SD. FIG. 2C shows the same as FIG. 2B but examined for Wnt5a protein expression by immunoblotting.

FIGS. 3A-3F show Wnt5a restoration inhibits the growth of human EOC cells by antagonizing canonical Wnt/β-catenin signaling. FIG. 3A shows OVCAR5 cells that were transduced with a control or Wnt5a-encoding puromycin-resistant retrovirus. The infected cells were drug-selected with 3 μg/mL puromycin. Expression of Wnt5a mRNA in drug-selected cells was determined by qRT-PCR. FIG. 3B shows the same as FIG. 3A, but examined for expression of Wnt5a and β-actin in control or Wnt5a-infected OVCAR5 and primary HOSE cells by immunoblotting. Relative levels of Wnt5a expression was indicated on the basis of the densitometric analysis, using NIH ImageJ software. FIG. 3C shows the same as FIG. 3A, but equal number (5,000) of drug-selected control (open triangles and dotted line) or Wnt5a-infected cells (open circles and solid line) were cultured on plastic plates for 4 days, and the number of cells was counted [control±SD or Wnt5a±SD (n=3); Student's t test was used for calculating P value] at day 1 (6,666±1,258 vs. 5,000±1,000; P=0.1469), day 2 (14,583±954 vs. 9,583±3,463; P=0.084), day 3 (41,250±6,538 vs. 14,750±2,787; *, P=0.0038), and day 4 (83,055±8,978 vs. 35,416±2,055; **, P=0.001). Mean of 3 independent experiments with SD and linear regression. FIG. 3D shows the same as FIG. 3C, but grown under anchorage-independent condition in soft agar. The number of colonies was counted 2 weeks after initial inoculation. Mean of 3 independent experiments with SD. FIG. 3E, same as FIG. 3A, but examined for the levels of soluble β-catenin and β-actin expression by immunoblotting. NT, nontreated. FIG. 3F shows the same as FIG. 3A, but examined for expression of indicated β-catenin target genes by qRT-PCR. Expression of β-2-microglobulin was used to normalize the expression of indicated genes. *, P=0.0095; **, P=0.0012; and ***, P=0.0286 compared with controls.

FIGS. 4A-4F show that Wnt5a promotes senescence of primary HOSE cells. FIG. 4A shows young proliferating primary HOSE cells were passaged to senescence (after 7 population doublings). Expression of SA-β-gal activity was measured in young and naturally senescent primary HOSE cells. FIG. 4B shows the same as FIG. 4A. Quantitation of SA-β-gal-positive cells. **, P<0.001. FIG. 4C shows the same as FIG. 4A, but mRNA was isolated and examined for Wnt5a expression by qRT-PCR. Expression of B2M was used as a control. *, P=0.003. FIG. 4D shows young primary HOSE cells were transduced with retrovirus encoding human Wnt5a gene or a control. Expression of Wnt5a in indicated cells was determined by qRT-PCR. Expression of B2M was used as a control. FIG. 4E shows the same as FIG. 4D, but stained for expression of SA-β-gal activity in drug-selected cells. FIG. 4F shows a quantitation of FIG. 4E. Mean of 3 independent experiments with SD. *, P<0.05.

FIGS. 5A-5E show Wnt5a restoration triggers cellular senescence in human EOC cells. FIG. 5A shows control and Wnt5a-expressing OVCAR5 EOC cells were stained with antibodies to HIRA and PML. Arrows point to examples of colocalized HIRA and PML bodies. Bar, 10 μm. FIG. 5B shows a quantitation of FIG. 5A. A total of 200 cells from control and Wnt5a-expressing cells were examined for HIRA and PML colocalization. Mean of 3 independent experiments with SD. FIG. 5C shows the same as FIG. 5A, but examined for pRB and GAPDH expression. FIG. 5D shows the same as FIG. 5C, but examined for pRBpS780 and GAPDH expression. FIG. 5E shows the same as FIG. 5A, but examined for SA-β-gal activity. FIG. 5F shows a quantitation of FIG. 5E. Mean of 3 independent experiments with SD.

FIGS. 6A-6F show Wnt5a restoration inhibits tumor growth and promotes senescence of human EOC cells in vivo. FIG. 6A shows OVCAR5 cells were transduced with luciferase-encoding hygromycin-resistant retrovirus together with a control or Wnt5a-encoding puromycin-resistant retrovirus. Drug-selected cells were unilaterally injected into the periovarian bursa sac of the female immunocompromised mice (n=6 for each of the groups). The radiance of luciferase bioluminescence, an indicator of the rate for tumor growth, was measured every 5 days from day 10 until day 30 by using the IVIS imaging system. Shown are images taken at day 10 and day 30, respectively. FIG. 6B shows a quantitation of tumor growth from injected OVCAR5 cells expressing Wnt5a or control at indicated time points. *, P=0.038 compared with controls. FIG. 6C shows that following tumor dissection, expression of Wnt5a in tumors formed by control or Wnt5a-expressing OVCAR5 EOC cells was determined by immunohistochemical staining against Wnt5a (magnification, 40×). Bar, 50 μm. FIG. 6D shows the same as FIG. 6C, but examined for expression of Ki-67, a marker of cell proliferation (magnification, 40×). Bar, 50 μm. FIG. 6E shows a quantitation of FIG. 6D. *, P=0.008 compared with controls. FIG. 6F shows expression of SA-β-gal activity was examined on sections of fresh-frozen tumors formed by OVCAR5 cells expressing control or Wnt5a (magnification, 40×). Bar, 100 μm. FIG. 6G shows quantitation of FIG. 6F. *, P=0.003 compared with controls. Arrow points to an example of SA-β-gal positive cells.

FIGS. 7A and 7B show confirmation of Wnt5a antibody specificity. FIG. 7A shows OVCAR5 cells were infected with Wnt5a encoding retrovirus together with a lentivirus encoding an shRNA to the human Wnt5a gene (shWnt5a) or control. Drug-selected cells were examined for Wnt5a and GAPDH expression by immunoblotting. FIG. 7B shows serial sections of xenograft tumors formed by OVCAR5 EOC cells ectopically expressing Wnt5a were subjected to immunohistochemical staining using an anti-Wnt5a antibody or an isotype matched IgG control (10×). Bar=100 μm.

FIGS. 8A-8D show Wnt5a restoration inhibits the growth of PEO1 EOC cells by antagonizing canonical Wnt signaling. FIG. 8A shows PEO1 cells that were transduced with a control- or Wnt5a-encoding puromycin resistant retrovirus. The infected cells were drug-selected with 1 μg/ml puromycin for 3 days. Expression of Wnt5a and GAPDH in drug-selected cells was determined by immunoblotting. FIG. 8B shows the same as FIG. 8A but an equal number of drug-selected control and Wnt5a infected PEO1 cells were cultured on plastic plate for 5 days and the number of cells were counted using trypan blue exclusion assay. Mean of three independent experiments with SD. FIG. 8C shows the same as FIG. 8B but grown under anchorage-independent conditions in soft-agar. The number of colonies were counted 2 weeks after initial inoculation. Mean of 3 independent experiments with SD. FIG. 8D shows the same as FIG. 8A, but examined for soluble β-catenin expression by immunoblotting.

FIGS. 9A-9F show the HIRA/PML pathway is activated during senescence of primary human ovarian surface epithelial (HOSE) cells induced by oncogenic-RAS. FIG. 9A shows primary HOSE cells were transduced with control or H-RAS^{G12V} encoding puromycin resistant retrovirus. Expression of RAS and β-actin were examined in drug-selected cells by immunoblotting. FIG. 9B shows the same as FIG. 9A but stained for SA-β-gal activity, a marker of cellular senescence. Percentage of SA-β-gal positive cells is indicated (mean of three independent experiments with SD). FIG. 9C shows the same as FIG. 9A but stained for HIRA and PML using indicated antibodies. Arrow points to an example of colocalized HIRA foci and PML body. Bar=10 μm. FIG. 9D shows the quantitation of FIG. 9C. The number of HIRA/PML foci positive cells was quantified from 100 cells from each indicated group. Mean of three independent experiments with SD. FIG. 9E shows primary HOSE cells were labeled with 10 μM BrdU for 1 hour and stained with indicated antibodies. Arrow points to an example of HIRA foci positive and BrdU negative cell and asterisk labels an example of HIRA foci negative and BrdU positive cells. Bar=10 μm. FIG. 9F shows a quantitation of FIG. 9E, BrdU positive cells were counted in primary HOSE cells with or without HIRA foci.

FIGS. 10A-10D show Wnt5a restoration triggers senescence in human EOC cells. FIG. 10A shows PEO1 EOC cells were infected with control or Wnt5a encoding puromycin-resistant retrovirus. The infected cells were drug-selected using 1 μg/ml of puromycin. Drug-selected cells were stained with antibodies to HIRA and PML. Arrows point to examples of co-localized HIRA and PML bodies. Bar=10 μm. FIG. 10B shows a quantitation of FIG. 10A. Mean of three independent experiments with SD. FIG. 10C shows the same as FIG. 10A but stained for SA-β-gal activity. FIG. 10D shows a quantitation of FIG. 10C. Mean of three independent experiments with SD.

FIG. 11 shows validation of an orthotopic EOC mouse model. OVCAR5 human EOC cells were injected into peri-ovarian bursa sac of female immuno-compromised mice. Shown is an example of a xenografted tumor together with a mouse ovary and an intact bursa sac (arrows), (stained with Hematoxylin and Eosin; H&E). Bar=200 μm.

### DETAILED DESCRIPTION OF THE INVENTION

Various terms relating to aspects of the present invention are used throughout the specification and claims. Such terms are to be given their ordinary meaning in the art, unless otherwise indicated. Other specifically defined terms are to be construed in a manner consistent with the definition provided herein.

As used herein, the singular forms “a,” “an,” and “the” include plural referents unless expressly stated otherwise.

Subject and patient are used interchangeably. A subject may be any animal, including mammals such as companion animals, laboratory animals, and non-human primates. Human beings are preferred.

Express, expressed, or expression of a nucleic acid molecule comprises the biosynthesis of a gene product. These terms encompass, without limitation, the transcription of a gene into RNA, the translation of RNA into a protein or polypeptide, and all naturally occurring post-transcriptional and post-translational modifications thereof.

Inhibiting includes reducing, decreasing, blocking, preventing, delaying, inactivating, desensitizing, stopping, knocking down (e.g., knockdown), and/or downregulating the biologic activity or expression of a molecule or pathway of interest.

It has been observed in accordance with the invention that in epithelial-derived ovarian cancers, the non-canonical Wnt ligand, Wnt5a, is often lost in high grade serous cancers, and that Wnt5a expression may be used to predict overall survival. In addition, it was observed that restoration of Wnt5a expression antagonizes canonical Wnt signaling by promoting the loss of active β-catenin, which results in downregulation of TCF/LEF target genes. In both in vitro and in vivo models of ovarian cancers, inhibition of canonical Wnt signaling in epithelial ovarian carcinoma (EOC) cells with Wnt5a expression resulted in a significant inhibition of proliferation. Importantly, the cause of this growth inhibition was a result of the induction of cellular senescence. These findings demonstrate that restoring Wnt5a expression may promote senescence in cancer cells, and that Wnt5a expression levels may serve as a marker for particular cancer stages.

Accordingly the invention features methods for characterizing the stage of an epithelial cancer in a subject. In some aspects, the methods comprise comparing the level of expression of a nucleic acid encoding Wnt5a determined from a tissue sample obtained from a subject with one or more reference values for the expression level of the nucleic acid encoding Wnt5a, said reference values preferably being obtained or derived from the same tissue as the tissue sample obtained from the subject, and characterizing the stage of the epithelial cancer based on the comparison. In preferred aspects, the comparing step is carried out using a processor capable of comparing, and preferably programmed to compare, the determined levels and reference values. The methods may comprise determining the level of expression of the nucleic acid encoding Wnt5a in the tissue sample obtained from a subject. The methods may comprise obtaining a tissue sample from a subject.

The nucleic acid encoding Wnt5a may be an mRNA, or may be a cDNA obtained from an mRNA. Techniques for determining the expression levels of such nucleic acids are well known in the art, and the particular technique used in accordance with the methods is not critical.

In some aspects, the methods comprise comparing the concentration of Wnt5a protein determined from a tissue sample obtained from a subject with one or more reference values for the concentration of the Wnt5a protein, said reference values preferably being derived or obtained from the same tissue as the tissue sample obtained from the subject, and characterizing the stage of the epithelial cancer based on the comparison. In preferred aspects, the comparing step is carried out using a processor capable of comparing, and preferably programmed to compare, the determined concentrations and reference values. The methods may comprise determining the concentration of Wnt5a protein in the tissue sample obtained from a subject. The methods may comprise obtaining a tissue sample from a subject.

The Wnt5a protein may be a fragment of the Wnt5a protein. Techniques for determining protein concentrations are well known in the art, and the particular technique used in accordance with the methods is not critical.

The U.S. National Cancer Institute classifies cancer according to four basic stages: Stage I, Stage II, Stage III, and Stage IV, based on the TNM scoring system (Primary Tumor, Regional Lymph Nodes, and Distant Metastasis). Thus, the methods, whether based on Wnt5a nucleic acid levels or protein concentrations, may be used to characterize the stage of the epithelial cancer as stage I epithelial cancer, stage II epithelial cancer, stage III epithelial cancer, or stage IV epithelial cancer. The methods may be used to determine that the subject does not have any epithelial cancer. The methods may be carried out in vitro, in vivo, or in situ.

The reference values may comprise one or more expression levels for the nucleic acid encoding Wnt5a, for example, mRNA expression levels. For example, the expression levels may be expression levels associated with stage I epithelial cancer, expression levels associated with stage II epithelial cancer, expression levels associated with stage III epithelial cancer, expression levels associated with stage IV epithelial cancer, or expression levels in a healthy subject, for example, a subject that does not have an epithelial cancer at any stage.

The reference values may comprise one or more concentration levels for Wnt5a protein in a particular tissue. For example, the Wnt5a protein concentration may be a concentration associated with stage I epithelial cancer, a concentration associated with stage II epithelial cancer, a concentration associated with stage III epithelial cancer, a concentration associated with stage IV epithelial cancer, or a concentration in a healthy subject, for example, a subject that does not have an epithelial cancer at any stage.

The methods may be used to characterize the stage of any epithelial cancer, particularly epithelial cancers in which cell proliferation or in which defects in normal senescence relate to the expression of Wnt5a, or which may otherwise be characterized by measuring Wnt5a gene expression levels or protein concentrations. Non-limiting examples of epithelial cancers include epithelial cancer of the ovary, rectum, breast (for example, estrogen receptor negative), prostate, pancreas, esophagus, bladder, liver, uterus, pancreas, or brain, as well as colorectal epithelial cancer, acute lymphoblastic leukemia, or esophageal squamous cell cancer. The methods are preferably used for characterizing the stage of ovarian cancer. Thus, the reference values may comprise the Wnt5a expression levels or protein concentrations for one or more of these types of epithelial cancers, and preferably for ovarian cancer. The tissue sample may thus be obtained from any tissue in which Wnt5a expression or concentration may be measured and correlated with cancer stage. In preferred aspects, the tissue sample is obtained from tumor tissue or a tissue suspected to be a tumor or neoplastically transformed. Healthy tissue may be obtained and screened as an internal control for the subject.

In some aspects, the methods may optionally comprise determining the subject's prognosis based on the comparison. A prognosis may relate to, or be measured according to any time frame. For example, the prognosis may comprise a substantial likelihood of mortality within about five years. The prognosis may comprise a substantial likelihood of mortality within about three years. The prognosis may comprise a substantial likelihood of mortality within about two years. The prognosis may comprise a substantial likelihood of mortality within about one year. In some aspects, the prognosis may comprise an about two to about five year range of time. The prognosis may comprise an about three to about five year range of time. The prognosis may comprise an about three to about ten year range of time. The prognosis may comprise an about five to about ten year range of time. Time frames may be shorter than one year or may be longer than five years. Time frames may vary according to clinical standards, or according to the needs or requests from the patient or practitioner.

Optionally, the methods may comprise treating the subject with a regimen capable of improving the prognosis of a patient having an epithelial cancer. The regimen may be capable of improving the prognosis of a patient having a specific epithelial cancer such as an epithelial cancer of the ovary, colon, rectum, breast, prostate, pancreas, esophagus, bladder, liver, uterus, or brain, and improving the prognosis of a patient having the epithelial cancer that the subject has. The regimen may be capable of preventing, inhibiting, or otherwise slowing the development of the cancer. For subjects determined to have an early stage cancer, for example, stage I or stage II cancer, the methods may comprise treating the subject with a regimen capable of preventing, inhibiting, or otherwise slowing the advancement of the cancer to a later stage.

The regimen may be tailored to the specific characteristics of the subject, for example, the age, sex, or weight of the subject, the type or stage of the cancer, and the overall health of the subject. The regimen may comprise one or more of surgery, radiation therapy, proton therapy, ablation therapy, hormone therapy, chemotherapy, immunotherapy, stem cell therapy, follow up testing, diet management, vitamin supplementation, nutritional supplementation, exercise, physical therapy, transplantation, reconstruction, psychological counseling, social counseling, education, and regimen compliance management. Suitable treatments for ovarian cancer include administering to the subject an effective amount of platinum and/or paclitaxel, as well as surgical debulking.

In some preferred aspects, the treatment regimen comprises enhancing the expression of Wnt5a in the subject. Enhancing the expression of Wnt5a may occur at the genetic level, utilizing suitable gene therapy methodologies to increase the expression of the Wnt5a gene, or to reverse or inhibit the downregulation of the Wnt5a gene. The regimen may comprise activating the histone repressor A (HIRA) pathway in the subject. The regimen may comprise antagonizing the Wnt canonical signaling pathway in the subject.

Enhancing the expression of Wnt5a may be carried out pharmaceutically, for example, by administering to the subject an effective amount of a compound or composition that enhances the expression of the Wnt5a gene, or otherwise reverses or inhibits Wnt5a downregulation. An example of a compound that inhibits Wnt5a downregulation is the DNA demethylation drug, 5-Aza-cytadine. In some aspects, the treatment regimen comprises administering to the subject a therapeutically effective amount of the Wnt5a protein, or biologically active fragment thereof. Biologically active fragments include the Wnt5a-derived hexapeptide, Foxy-5. (Säfholm A et al. (2008) Clin. Cancer Res. 14:6556-63).

The steps of the methods, including any optional steps, may be repeated after a period of time, for example, as a way to monitor a subject's health and prognosis. Repeating the methods may be used, for example, to determine if a subject has advanced from an early stage epithelial cancer to a later stage epithelial cancer. Repeating the methods may be used, for example, to determine if the patient's prognosis has improved based on a particular treatment regimen, or to determine if adjustments to the treatment regimen should be made to achieve improvement or to attain further improvement in the patient's prognosis. The methods may be repeated at least one time, two times, three times, four times, or five or more times. The methods may be repeated as often as the patient desires, or is willing or able to participate.

The period of time between repeats may vary, and may be regular or irregular. In some aspects, the methods are repeated in three month intervals. In some aspects, the methods are repeated in six month intervals. In some aspects, the methods are repeated in one year intervals. In some aspects, the methods are repeated in two year intervals. In some aspects, the methods are repeated in five year intervals. In some aspects, the methods are repeated only once, which may be about three months, six months, twelve months, eighteen months, two years, three years, four years, five years, or more from the initial assessment.

The invention also features systems that may be used, for example, to carry out the methods. In some aspects, a system for characterizing the stage of an epithelial cancer comprises a data structure comprising one or more reference values comprising one or more values of Wnt5a nucleic acid expression levels associated with stage I epithelial cancer, expression levels associated with stage II epithelial cancer, expression levels associated with stage III epithelial cancer, expression levels associated with stage IV epithelial cancer, or expression levels in a healthy subject, and a processor operably connected to the data structure. The processor is capable of comparing, and preferably programmed to compare, the level of expression of a nucleic acid encoding Wnt5a determined from a tissue sample obtained from a subject and reference values. The nucleic acid encoding Wnt5a may be an mRNA or a cDNA obtained from mRNA.

In some aspects, a system for characterizing the stage of an epithelial cancer comprises a data structure comprising one or more reference values comprising one or more values of Wnt5a protein concentration levels associated with stage I epithelial cancer, concentration levels associated with stage II epithelial cancer, concentration levels associated with stage III epithelial cancer, concentration levels associated with stage IV epithelial cancer, or concentration levels in a healthy subject, and a processor operably connected to the data structure. The processor is capable of comparing, and preferably programmed to compare, the Wnt5a protein concentration levels determined from a tissue sample obtained from a subject and reference values.

In the systems, a processor may be a computer processor. A computer may comprise the processor. The systems may comprise a computer network connection, including an Internet connection. The systems may comprise computer readable media comprising executable code to cause a processor to carry out desired operations such as measurements, determinations, comparisons or recommendations.

The systems may be used to characterize the stage of any epithelial cancer, particularly epithelial cancers in which cell proliferation or in which defects in normal senescence relate to the expression of Wnt5a, or which may otherwise be characterized by measuring Wnt5a gene expression levels or protein concentrations. Non-limiting examples of epithelial cancers include epithelial cancer of the ovary, colon, rectum, breast, prostate, pancreas, esophagus, bladder, liver, uterus, or brain.

The systems may optionally comprise a processor capable of determining, and preferably programmed to determine the level of expression of a nucleic acid encoding Wnt5a obtained from a subject. The nucleic acid may be isolated from the tissue, or in the tissue. The processor may thus be operably connected to systems or devices that detect nucleic acids.

The systems may optionally comprise a processor capable of determining, and preferably programmed to determine the concentration levels of Wnt5a protein obtained from a subject. The protein may be isolated from the tissue, or in the tissue. The processor may thus be operably connected to systems or devices that detect polypeptides.

The systems may comprise an input for accepting a determined level of expression of a nucleic acid encoding Wnt5a obtained from the subject and/or an input for accepting a determined concentration of Wnt5a protein obtained from the subject. The systems may comprise an output for providing results of a comparison, such as a comparison of a determined nucleic acid level or protein concentration with one or more reference values to a user such as the subject, or a technician, or a medical practitioner.

The invention also features computer readable media that may be used, for example, in accordance with the systems and/or to carry out the methods. The computer readable media may comprise a processor, which may be a computer processor.

In some aspects, a computer readable medium comprises executable code for causing a programmable processor to compare the expression level of a nucleic acid encoding Wnt5a in a tissue sample obtained from a subject with one or more reference values comprising one or more Wnt5a nucleic acid expression level values associated with stage I epithelial cancer, expression levels associated with stage II epithelial cancer, expression levels associated with stage III epithelial cancer, expression levels associated with stage IV epithelial cancer, or expression levels in a healthy subject. In some aspects, a computer readable medium comprises executable code for causing a programmable processor to compare the concentration of Wnt5a protein in a tissue sample obtained from a subject with one or more reference values comprising one or more Wnt5a protein concentration levels associated with stage I epithelial cancer, concentration levels associated with stage II epithelial cancer, concentration levels associated with stage III epithelial cancer, concentration levels associated with stage IV epithelial cancer, or concentration levels in a healthy subject. The reference values may be values for any epithelial cancer, non-limiting examples of which include epithelial cancers of the ovary, colon, rectum, breast, prostate, pancreas, esophagus, bladder, liver, uterus, or brain.

The computer readable media may optionally comprise executable code for causing a programmable processor to determine a prognosis for an epithelial cancer patient based on a comparison of a determined level of expression of a nucleic acid encoding Wnt5a or a determined concentration of the Wnt5a protein with appropriate reference values such as those described herein. The computer readable media may optionally comprise executable code for causing a programmable processor to recommend a treatment regimen for treating an epithelial cancer patient. The treatment regimen may relate to the particular type of epithelial cancer, and may relate to the particular stage of the epithelial cancer. The treatment regimen may comprise a treatment regimen described or exemplified in this specification. The executable code may cause a programmable processor to customize a treatment regimen.

The invention also features methods for inducing senescence in an epithelial cancer cell. The methods may be carried out in vitro, in situ, or in vivo.

In some aspects, the methods may comprise enhancing the level of expression of the Wnt5a gene in an epithelial cancer cell. Enhancing the level of expression may include reversing or inhibiting downregulation of the Wnt5a gene and/or facilitating upregulation of the Wnt5a gene. Enhancing the level of expression of Wnt5a may be capable of activating the HIRA pathway in the cell, and/or antagonizing the Wnt canonical signal pathway in the cell, and/or antagonizing the beta-catenin pathway in the cell.

In some aspects, the methods may comprise contacting an epithelial cancer cell with an amount of the Wnt5a protein, or a biologically active fragment thereof, effective to induce senescence in the cell. The Wnt5a protein or biologically active fragment thereof may comprise a fusion protein, for example, a fusion with an antibody in order to facilitate targeting of the Wnt5a protein to the cell of interest, or a fusion with a protein to facilitate entry of the Wnt5a protein into the cell.

The methods may be used to induce senescence in any epithelial cancer cell. Non-limiting examples of epithelial cancer cells include epithelial cancer cells of the ovary, colon, rectum, breast, prostate, pancreas, esophagus, bladder, liver, uterus, or brain. Epithelial cancer cells of the ovary are preferred.

The following examples are provided to describe the invention in greater detail. They are intended to illustrate, not to limit, the invention.

### Example 1

### Materials and Methods

Cell culture, soft agar assay and inducible Wnt5a expression human EOC cell lines. Primary human ovarian surface epithelial (HOSE) cells were isolated and cultured. The protocol was approved by the Fox Chase Cancer Center institutional review board. All experiments were performed in multiple batches of primary HOSE cells. Human epithelial ovarian carcinoma cell (EOC) lines, A1847, A2780, OVCAR3, OVCAR5, OVCAR10, PEO1, UPN289 and SKOV3 were donated. All human EOC cell lines were cultured according to ATCC guidelines in RPMI 1640 medium supplemented with 10% fetal bovine serum (FBS). For anchorage-independent soft agar assay, 3500 cells were resuspended in 0.35% low melt agarose melted in RPMI 1640 medium supplemented with 10% FBS, and inoculated on top of 0.6% low melt agarose base in six well plates. After 2 weeks of culture, the plates were stained with 0.005% crystal violet and the number of colonies was counted using a dissecting microscope. Inducible Wnt5a-expressing PEO1 and OVCAR5 cell lines were established using Retro-X™-Tet-On® inducible system (Clontech Laboratories, Inc., Mountain View, Calif.) according to the manufacture's instructions. To induce Wnt5a expression, 1 μg/ml final concentration of doxycycline was supplemented in the cell culture medium.

Retrovirus and lentivirus infections. The following retrovirus construct was utilized: pBabe-puro and was purchased from Addgene, and a pBABE-Wnt5a construct was generated using a standard cloning protocol. Retrovirus packaging was performed using Pheonix packaging cells. To increase infection efficacy, double virus infection was performed. For drug-selection, 1 μm/ml of puromycin was used for the PEO1 human EOC cell line, whereas 3 μg/ml of puromycin was used for the OVCAR5 human EOC cell line.

RT-PCR, qRT-PCR and immunoblotting. RNA from cultured primary HOSE or human EOC cells was isolated using Trizol (Invitrogen) according to manufacture's instruction. For qRT-PCR, Trizol®—(Molecular Research Center, Inc., Cincinnati, Ohio) isolated RNA was further purified using a RNeasy® mini kit (Qiagen GmbH, Germany). The Wnt5a primers used for qRT-PCR were purchased from Applied Biosystems. Housekeeping β-2-microglobulin or β-actin mRNA expression was used to normalize the Wnt5a mRNA expression. Soluble β-catenin was extracted using a buffer that consisting of 10 mM pH 7.5 Tris-HCl, 0.05% NP-40, 10 mM NaCl, 3 mM MgCl₂, 1 mM EDTA and proteinase inhibitors (Roche). The following antibodies were used for immunoblotting: goat anti-Wnt5a (R&D systems), mouse anti-GAPDH (Millipore/Chemicon), mouse anti-β-catenin (BD Bioscience), anti-p53, mouse anti-Rb and mouse anti-β-actin (Sigma Chemical Co.), and rabbit anti-pRBpS780 (Cell Signaling).

Immunofluorescence staining, and SA-β-gal staining. Indirect immunofluorescence staining was performed. The following antibodies were used for immunofluorescence: a cocktail of mouse anti-HIRA monoclonal antibodies (WC19, WC117 and WC119, 1:10) and a rabbit anti-PML antibody (Chemicon, 1:5000). Images were captured using a DS-Qilmc camera on a Nikon Eclipse 80i microscope, and processed using NIS-Elements BR3.0 software (Nikon). SA-β-gal staining was performed.

Human ovarian specimens and immunohistochemistry. Ovarian tumor microarray and normal human ovary specimens were obtained from an in-house Biosample Repository Core Facility (BRCF). Histopathology of the selected specimens on the tumor microarrays was provided by BRCF. Histopathology of the selected specimens on the tumor microarrays was provided by BRCF. Immunohistochemistry (IHC) was conducted by using goat anti-Wnt5a polyclonal antibody (R&D Systems) and mouse anti-Ki-67 (Dako) with a DAKO EnVision System and the Peroxidase (DAB) kit (DAKO Corporation) following the manufacturer's instructions. Wnt5a staining intensity was scored in a four-tier grading system in a double blinded manner by a board certified pathologist.

Xenograft Mouse Study. The protocol was approved by the FCCC Institutional Animal Care and Use Committee. OVCAR5 cells were infected with a luciferase-encoding retrovirus (hygro-pWZL-luciferase) and infected cells were selected with 50 mg/mL hygromycin. Drug-selected cells were then infected with control or Wnt5a-encoding retrovirus and subsequently selected with 3 mg/mL puromycin and 50 mg/mL hygromycin. A total of 3×10⁶ drug-selected cells were unilaterally injected into the ovarian bursa sac of immunocompromised mice (6 mice per group). From day 10 postinfection, tumors were visualized by injecting luciferin (intraperitoneal, 4 mg/mice) resuspended in PBS and imaged with an IVIS Spectrum imaging system every 5 days until day 30. Images were analyzed by Live Imaging 4.0 software. At day 30, tumors were surgically dissected and either fixed in 10% formalin or fresh-frozen in Optimal Cutting Temperature compound (Tissue-Tek). Sections of the dissected tumors were processed in-house.

Statistical analysis. Quantitative data are expressed as mean±SD, unless otherwise indicated. ANOVA with Student's t test was used to identify significant differences in multiple comparisons. The Pearson chi-squared test was used to analyze the relationship between categorical variables. Overall survival was defined as the time elapsed from the date of diagnosis and the date of death from any cause or the date of last follow-up. Kaplan-Meier survival plots were generated and comparisons were made by using the log-rank sum statistic. For all statistical analyses, the level of significance was set at 0.05.

### Example 2

### Results

Wnt5a is Expressed at Significantly Lower Levels in Human EOC Cell Lines and Primary Human EOCs Compared with Normal Human Ovarian Surface Epithelium or Fallopian Tube Epithelium

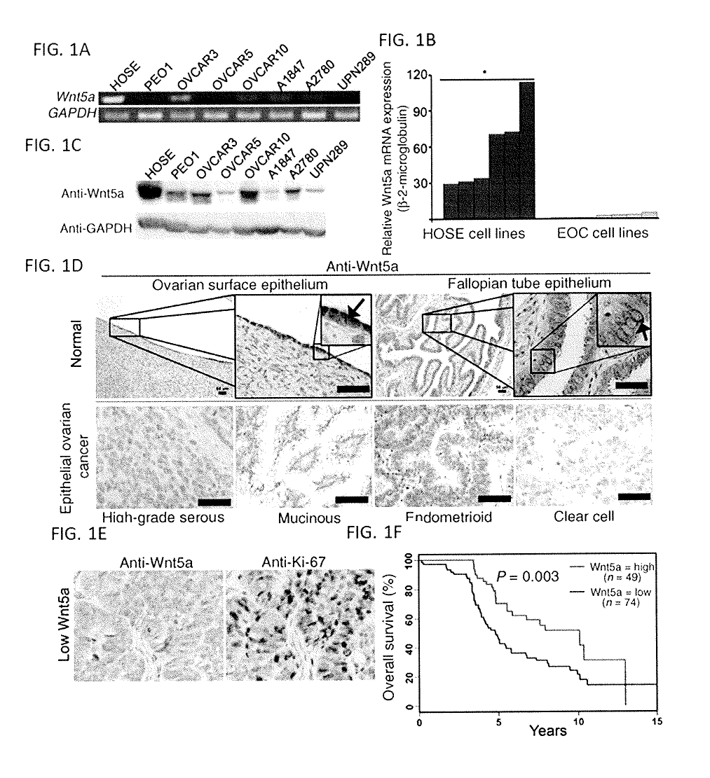

To determine Wnt5a expression in human EOC cell lines and primary HOSE cells, the relative Wnt5a mRNA levels were examined using semiquantitative RT-PCR. It was observed that Wnt5a mRNA levels were greatly diminished in human EOC cell lines compared with primary HOSE cells (FIG. 1A). This finding was further confirmed through qRT-PCR analysis of Wnt5a mRNA in multiple isolations of primary HOSE cells and human EOC cell lines, showing that the levels of Wnt5a mRNA were significantly lower in human EOC cell lines compared with primary HOSE cells (FIG. 1B; P=0.008). Consistently, Wnt5a protein levels were also lower in human EOC cell lines compared with primary HOSE cells as determined by immunoblotting (FIG. 1C). On the basis of these results, it was observed that Wnt5a is expressed at lower levels in human EOC cell lines compared with primary HOSE cells.

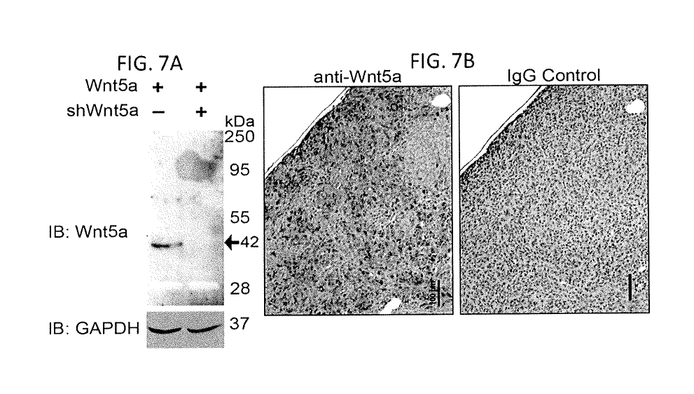

Whether the loss of Wnt5a expression found in human EOC cell lines was also observed in primary human EOCs was studied. Wnt5a expression was examined in 130 cases of primary human EOC specimens and 31 cases of normal human ovary with surface epithelium by IHC, using an antibody against Wnt5a (Table 1). In addition, recent evidence suggests that a proportion of high-grade serous EOC may arise from distant fallopian tube epithelium. Twenty eight cases of normal human fallopian tube specimens were included in the IHC analysis (Table 1). The specificity of the anti-Wnt5a antibody was confirmed in the study (FIG. 7). A single band at predicted molecular weight (˜42 kDa) was detected in OVCAR5 cells with ectopically expressed Wnt5a and was absent after expression of a short hairpin RNA to the human Wnt5a gene (shWnt5a), which effectively knocked down Wnt5a mRNA expression (FIG. 7A). In addition, Wnt5a staining was lost when primary anti-Wnt5a antibody was replaced with an isotype-matched IgG control (FIG. 7B).

TABLE 1

| Patient | Wnt5a Protein Expression | | | | |
|---|---|---|---|---|---|
| characteristics | Low (n) | High (n) | Total (n) | High (%) | P |
| Age (23-85 yrs, | | | | | |
| mean 59.2 yrs) | | | | | |
| <55 | 24 | 16 | 40 | 40.0% | |
| >55 | 52 | 33 | 85 | 38.8% | 0.900 |
| Unknown | 5 | 0 | 5 | | |
| Laterality | | | | | |
| Left | 22 | 14 | 36 | 38.9% | |
| Right | 12 | 9 | 21 | 42.9% | 0.957 |
| Bilaterality | 35 | 24 | 59 | 40.7% | |
| Undetermined | 12 | 2 | 14 | | |
| Histotype | | | | | |
| Epithelial ovarian | 81 | 49 | 130 | 37.7% | |
| cancer | | | | | |
| Type I | 16 | 21 | 37 | 56.8% | |
| Low Grace Serous | 1 | 1 | 2 | 50.0% | |
| Endometrioid | 4 | 9 | 13 | 69.2% | |
| Mucinous | 2 | 3 | 5 | 60.0% | |
| Clear | 5 | 4 | 9 | 44.4% | |
| Others | 4 | 4 | 8 | 50.0% | |
| Type II | | | | | 0.005* |
| High Grade Serous | 65 | 28 | 93 | 30.1% | |
| Normal epithelium | | | | | |
| Ovarian Surface | 13 | 18 | 31 | 58.1% | 0.039** |
| Epithelium | | | | | |
| Fallopian Tube | 5 | 23 | 28 | 82.1% | <0.001** |
| Ki-67 | | | | | |
| Low | 22 | 23 | 44 | 52.3 | |
| High | 51 | 24 | 75 | 32.0 | 0.038 |
| Undetermined | 7 | 3 | 11 | | |
| Tumor grade | | | | | |
| 1 | 3 | 7 | 10 | 70.0% | |
| 2 | 12 | 8 | 20 | 40.0% | |
| 3 | 64 | 31 | 95 | 32.6% | 0.086* |
| Undetermined | 2 | 3 | 5 | | |
| Tumor stage | | | | | |
| Stage 1/2 | 12 | 18 | 30 | 60.0% | |
| Stage 3/4 | 67 | 29 | 96 | 30.2% | 0.003*** |
| Undetermined | 2 | 2 | 4 | | |

P values generated from Pearson Chi-Square Test # Performed Fisher Exact Test
*Compared type I;
**compared with epithelial ovarian cancer;
***compared with Stage 1/2

As shown in Hg. 1D, in normal human ovarian surface epithelial cells and fallopian tube epithelial cells, both cytoplasm and cell membrane were positive for Wnt5a IHC staining (arrows, FIG. 1D). In contrast, Wnt5a staining in EOC cells was dramatically decreased (FIG. 1D). Expression of Wnt5a was scored as high (H-score≧30) or low (H score<30) on the basis of a histological score (H score; 26), which considered both intensity of staining and percentage of positively stained cells, as previously described. Wnt5a expression was scored as high in 58.1% (18/31) cases of normal human ovarian surface epithelium and 82.1% (23/28) cases of normal human fallopian tube epithelium (Table 1). In contrast, Wnt5a expression was scored as high in 37.7% (49/130) cases of primary human EOCs (Table 1). Statistical analysis revealed that Wnt5a was expressed at significantly lower levels in primary human EOCs compared with either normal human ovarian surface epithelium (P=0.039) or normal human fallopian tube epithelium (P<0.001; Table 1). On the basis of these studies, it was observed that Wnt5a is expressed at significantly lower levels in primary human EOCs compared with either normal human ovarian surface epithelium or fallopian tube epithelium.

Wnt5a expression negatively correlates with tumor stage and lower Wnt5a expression predicts shorter overall survival. The correlation between Wnt5a expression and clinical and pathologic features of human EOCs was examined. Significantly, there was a negative correlation between Wnt5a expression and tumor stage (P=0.003; Table 1). The majority of examined cases were high-grade serous subtypes that are usually of stage 3/4. In addition, the correlation between expression of Wnt5a and a marker of cell proliferation, Ki-67 (FIG. 1E) was examined. There was a significant negative correlation between Wnt5a expression and Ki-67 (P=0.038; Table 1). Whether Wnt5a expression based on H score might predict prognosis of EOC patients (High, H score≧30; Low, H score<30; n=123), for which long-term follow-up data were available was assessed. Significantly, lower Wnt5a expression correlated with shorter overall survival in the examined EOC patients (P=0.003; FIG. 1F). It was concluded that a lower level of Wnt5a expression correlates with tumor stage and predicts shorter overall survival in human EOC patients.

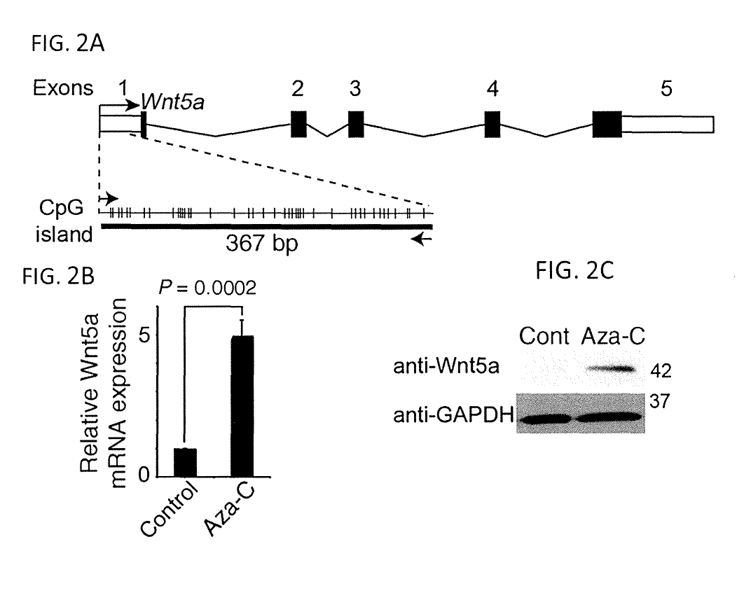

Wnt5a gene promoter hypermethylation contributes to its downregulation in human EOC cells. Wnt5a gene promoter hypermethylation has been implicated as a mechanism underlying its silencing in several types of human cancers. Consistently, Wnt5a gene promoter hypermethylation was observed in a number of human EOC cell lines (FIG. 2A; Table 2). Further supporting a role of promoter hypermethylation in suppression of Wnt5a expression, treatment with a DNA demethylation drug, Aza-C, in PEO1 EOC cells resulted in a significant increase in levels of both Wnt5a mRNA and protein (FIGS. 2B and C). It was observed that Wnt5a gene promoter hypermethylation contributes to its downregulation in human EOC cells.

TABLE 2

| EOC | Wnt5a Promoter |
|---|---|
| Cell Line | DNA Methylated |
| PEO-1 | Yes |
| A1847 | No |
| A2780 | Yes |
| OVCAR3 | No |
| OVCAR5 | Yes |
| OVCAR10 | Yes |
| SKOV3 | No |

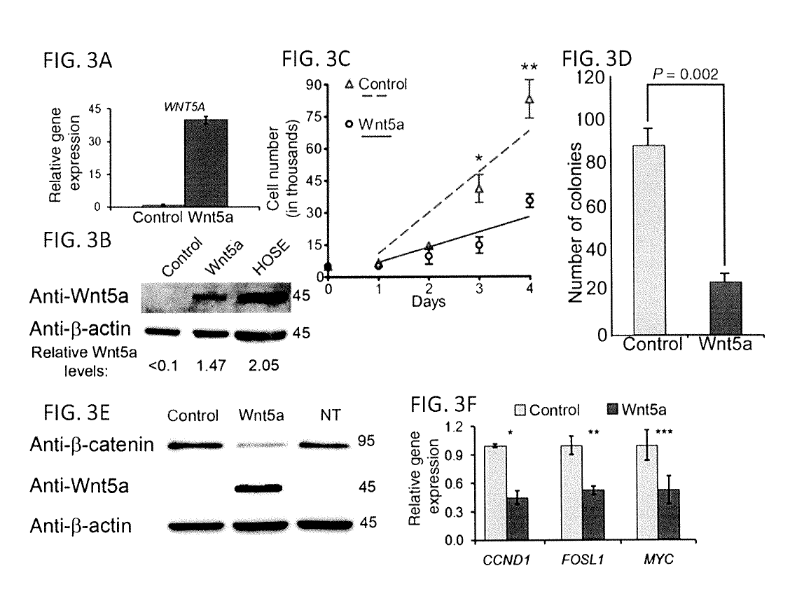

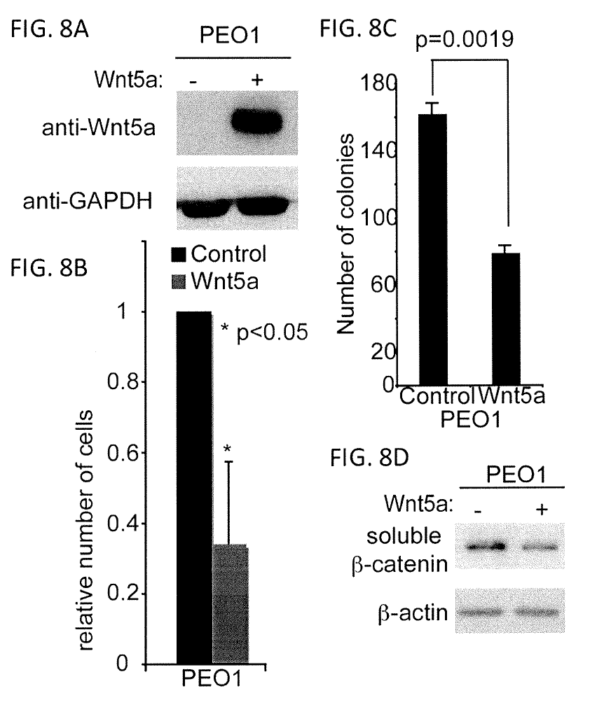

Wnt5a restoration inhibits the growth of human EOC cells by antagonizing the canonical Wnt/β-catenin signaling. The effects of Wnt5a reconstitution in human EOC cells were determined. Wnt5a expression was reconstituted in the OVCAR5 EOC cell line via retroviral transduction. Ectopically expressed Wnt5a was confirmed by both qRT-PCR and immunoblotting in OVCAR5 cells stably expressing Wnt5a or a vector control (FIGS. 3A and B). Of note, the levels of ectopically expressed Wnt5a in OVCAR5 cells were comparable with the levels observed in primary HOSE cells (FIG. 3B). Interestingly, Wnt5a reconstitution in OVCAR5 human EOC cells significantly inhibited both anchorage-dependent and anchorage-independent growth in soft agar compared with vector controls (FIGS. 3C and D). In addition, similar growth inhibition by Wnt5a reconstitution was also observed in the PEO1 human EOC cell line (FIG. 8A-C) suggesting that this effect is not cell line specific. On the basis of these results, it was observed that Wnt5a reconstitution inhibits the growth of human EOC cells in vitro.

Canonical Wnt signaling promotes cell proliferation and Wnt5a has been shown to antagonize the canonical Wnt/β-catenin signaling in certain cell contexts. Because Wnt5a expression inversely correlated with expression of Ki-67 (FIG. 1E; Table 1), a cell proliferation marker, it was hypothesized that Wnt5a would suppress the growth of human EOC cells by antagonizing canonical Wnt/β-catenin signaling. The effect of Wnt5a reconstitution on expression of markers of active Wnt/β-catenin signaling in human EOC cells, namely the levels of “active” soluble β-catenin and expression of β-catenin target genes such as CCND1, c-MYC, and FOSL1 was examined. A decrease in soluble β-catenin in Wnt5a-reconstituted OVCAR5 cells compared with vector controls (FIG. 3E) was observed. Consistently, a significant decrease in the levels of β-catenin target genes in these cells, namely CCND1 (P=0.0095), FOSL1 (P=0.0012), and c-MYC (P=0.0286; FIG. 3F) was also observed. Similar effects of Wnt5a reconstitution on expression of markers of active Wnt/β-catenin signaling (such as decreased levels of soluble β-catenin) were also observed in PEO1 human EOC cells (FIG. 8D), suggesting that this is not cell line specific. It was observed that Wnt5a suppresses the growth of human EOC cells by antagonizing canonical Wnt/β-catenin signaling in human EOC cells.

Wnt5a reconstitution drives cellular senescence in human EOC cells. The cellular mechanism whereby Wnt5a inhibits the growth of human EOC cells was determined. It was previously shown that suppression of canonical Wnt signaling promotes cellular senescence in primary human fibroblasts by activating the senescence-promoting histone repressor A (HIRA)/promyelocytic leukemia (PML) pathway. PML bodies are 20 to 30 dot-like structures in the nucleus of virtually all human cells. PML bodies are sites of poorly defined tumor suppressor activity and are disrupted in acute PML. PML has been implicated in regulating cellular senescence. For example, the foci number and size of PML bodies increase during senescence and inactivation of PML suppresses senescence. Activation of the HIRA/PML pathway is reflected by the recruitment of HIRA into PML bodies.

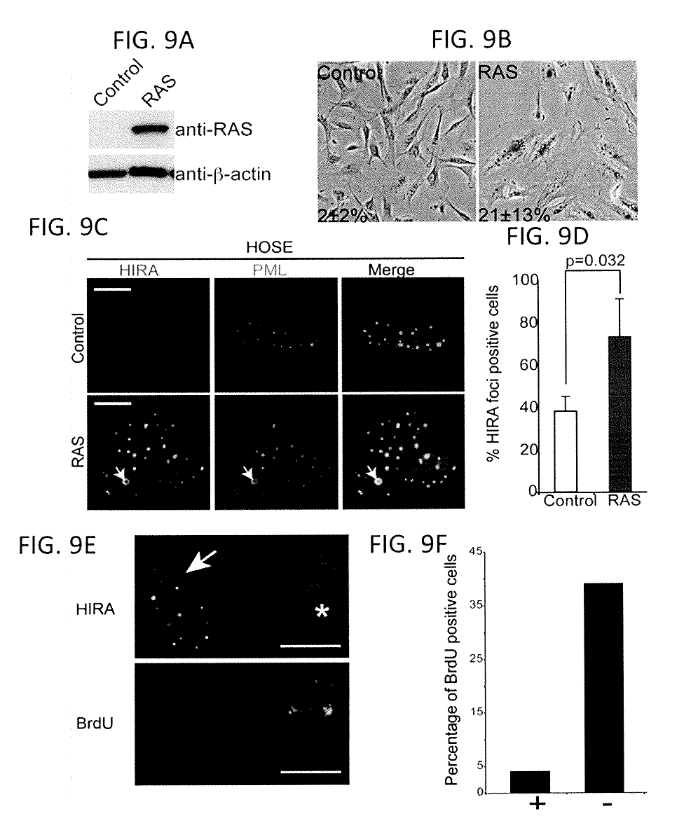

To determine whether Wnt5a reconstitution activates the HIRA/PML senescence pathway and induces senescence in EOC cells, the HIRA/PML pathway in human ovarian epithelial cells was examined. Ectopically expressing activated oncogenes (such as oncogenic RAS) is a standard approach for inducing senescence in a synchronized manner in primary human cells. Indeed, ectopic expression of oncogenic H-RAS^{G12V} induced senescence of primary HOSE cells, as was evident by an increase in SA-β-gal activity, a universal marker of cellular senescence (FIGS. 9A and 9B). Notably, the HIRA/PML pathway was activated during senescence of primary HOSE cells induced by oncogenic RAS, as evident by the relocalization of HIRA into PML bodies (FIGS. 9C and 9D). This result shows that the senescence-promoting HIRA/PML pathway is conserved in human ovarian epithelial cells. In addition, primary HOSE cells with HIRA foci displayed a marked decrease in BrdU incorporation, a marker of cell proliferation, compared with HIRA foci-negative cells (FIGS. 9E and 9F). This result is consistent with the idea that activation of the HIRA/PML pathway is directly correlated with senescence-associated cell growth arrest.

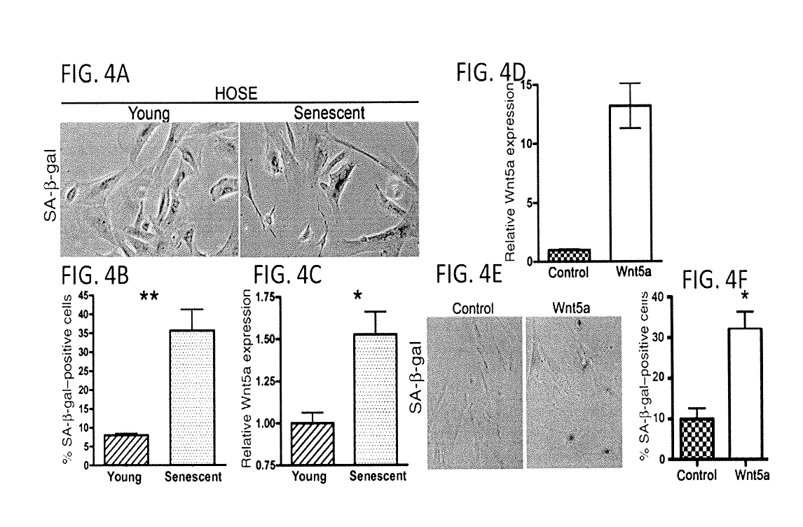

Whether Wnt5a expression is regulated during natural senescence of primary HOSE cells was determined. An increase in the levels of Wnt5a mRNA in senescent primary HOSE cells compared with young cells (FIG. 4A-C) was observed. In addition, it was found that ectopic Wnt5a induces senescence of primary HOSE cells (FIG. 4D-F). It was observed that Wnt5a plays a role in regulating senescence of primary HOSE cells.

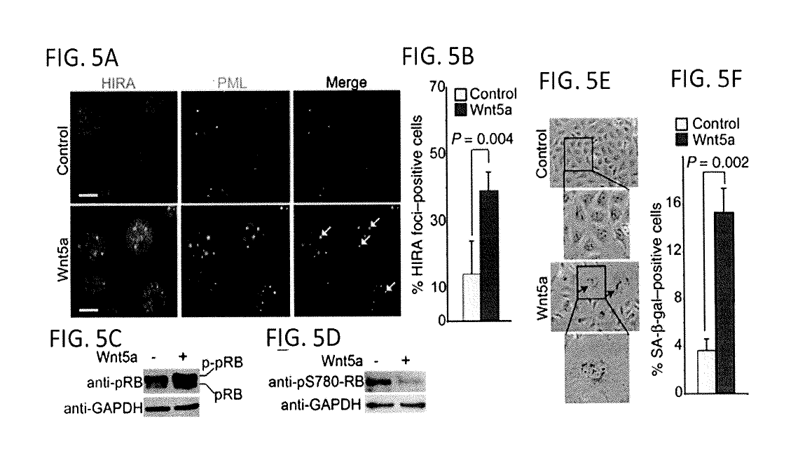

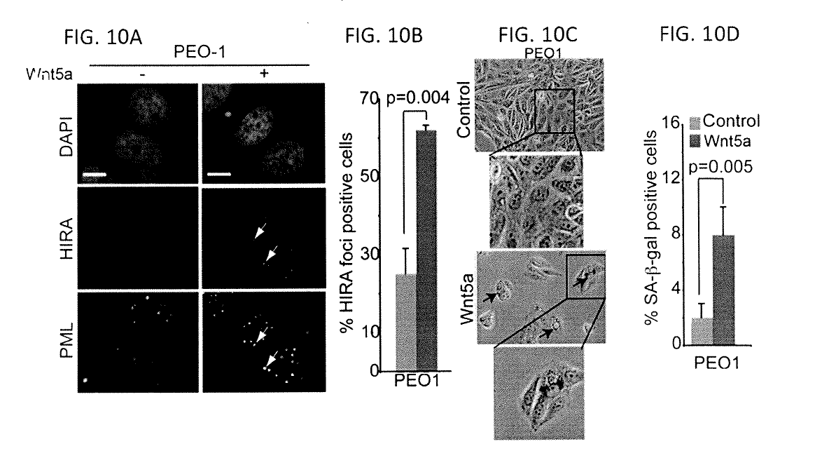

As Wnt5a antagonizes canonical Wnt signaling in human EOC cells (FIGS. 3E and F), whether Wnt5a restoration activates the senescence-promoting HIRA/PML pathway and induce senescence in human EOC cells was examined. The localization of HIRA in OVCAR5 EOC cells reconstituted with Wnt5a or vector control was examined. Notably, there was a significant increase in the percentage of cells with HIRA localized to PML bodies in Wnt5a restored human EOC cells compared with controls (FIGS. 5A and B; P=0.004). In addition, an increase in the number and size of PML bodies in the Wnt5a restored OVCAR5 EOC cells (FIG. 5A), which are also established markers of cellular senescence was also observed. Similarly, activation of the HIRA/PML pathway by Wnt5a restoration in PEO1 human EOC cells (FIGS. 10A and 10B), was also observed, suggesting that the observed effects are not cell line specific. It was observed that Wnt5a reconstitution activates the HIRA/PML senescence pathway.

The p53 and pRB tumor suppressor pathways play a key role in regulating senescence. Thus, whether activation of the HIRA/PML pathway depends on the p53 and pRB pathways was determined. Interestingly, p16^{INK4a}, the upstream repressor of pRB, is deleted in OVCAR5 human EOC cell line. In addition, the levels of total phosphorylated pRB were not decreased by Wnt5a, whereas the levels of cyclin D1/CKD4-mediated Serine 780 phosphorylation on pRB (pRBpS780) were decreased by Wnt5a (FIGS. 5C and D). Furthermore, p53 is null in OVCAR5 cells. It was concluded that activation of the HIRA/PML pathway is independent of the p53 and p16^{INK4a}.

Next, whether Wnt5a restoration induces SA-β-gal activity, a universal marker of cellular senescence was determined. Indeed, SA-β-gal activity was notably induced by Wnt5a reconstitution in both OVCAR5 and PEO1 human EOC cells compared with controls (FIGS. 5E and 5F; FIGS. 10C and 10D). On the basis of these results, it was observed that Wnt5a restoration induced senescence of human EOC cells by activating the HIRA/PML senescence pathway.

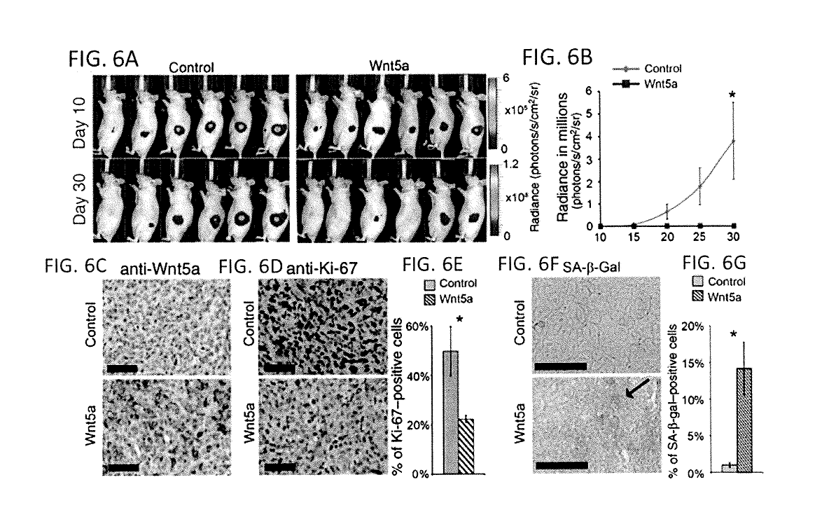

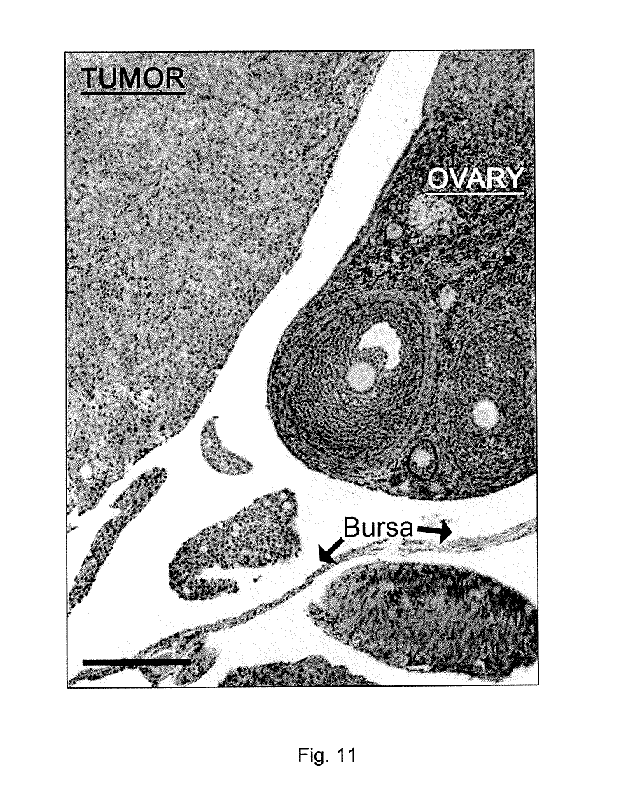

Wnt5a inhibits the growth of human EOC cells in vivo by inducing cellular senescence. Whether Wnt5a would mediate growth inhibition and induce senescence in vivo in an orthotopic EOC model in immunocompromised mice was determined. A luciferase gene was retrovirally transduced into control or Wnt5a-reconstituted OVCAR5 cells to monitor the cell growth in vivo via noninvasive imaging. These cells were injected unilaterally into the bursa sac covering the ovary in female immunocompromised mice (n=6 for each of the groups; FIG. 11). Tumor growth was monitored every 5 days starting at day 10 postinjection by measuring luciferase activity, and the growth of the tumor was followed for a total of 30 days (FIG. 6A). Wnt5a significantly suppressed the growth of xenografted OVCAR5 human EOC cells compared with controls (FIG. 6B; P<0.03). Consistently, following general pathologic examination during surgical dissection at day 30, it was observed that tumor sizes were notably smaller from mice injected with Wnt5a-reconstituted OVCAR5 cells compared with controls. The expression of ectopic Wnt5a was confirmed by IHC staining in sections from dissected tumors (FIG. 6C). Next, whether cell proliferation was suppressed by Wnt5a reconstitution in dissected tumors was determined. Toward this goal, the expression of Ki-67 by IHC was examined. A significant decrease in the number of Ki-67-positive cells in tumors formed by Wnt5a-reconstituted OVCAR5 cells compared with controls (FIGS. 6D and E) was observed. In addition, intensity of Ki-67 staining was also notably weaker in Ki-67-positive Wnt5A-reconstituted OVCAR5 cells than in control Ki-67-positive cells (FIG. 6D). It was observed that Wnt5a reconstitution inhibits the proliferation of human EOC cells in vivo in an orthotopic xenograft EOC model.

Whether the growth inhibition observed by Wnt5a reconstitution in vivo was due to induction of cellular senescence was investigated. Toward this goal, the expression of SA-β-gal activity in fresh sections of dissected tumors formed by OVCAR5 cells reconstituted with Wnt5a or control cells was examined. A significant increase in the number of cells positive for SA-β-gal activity in OVCAR5 cells reconstituted with Wnt5a compared with control tumors (FIGS. 6F and 6G; P=0.003) was observed. It was observed that Wnt5a reconstitution inhibits the growth of human EOC cells in vivo by inducing cellular senescence.

### Example 3

### Summary

The data show that restoration of Wnt5a signaling drives senescence of human EOC cells both in vitro and in vivo in an orthotopic mouse model of EOC (FIGS. 5 and 6). Restoring gene expression by gene therapy has had limited success. Therefore, restoring Wnt5a signaling via exogenous ligand could prove to be an alternative approach.

It is believed that these results are the first to show a role for Wnt5a in regulating senescence. Wnt5a activated the senescence-promoting HIRA/PML pathway in human EOC cells (FIG. 5A, FIG. 10A). In primary human cells, activation of HIRA/PML pathway is sufficient to drive senescence by facilitating epigenetic silencing of proliferation-promoting genes. The data are believed to be the first to show that the key HIRA/PML senescence pathway can be reactivated to drive senescence of human cancer cells.

Senescence induced by Wnt5a restoration in human EOC cells was independent of both the p53 and p16INK4a tumor suppressors, which implies that EOC cells that lack p53 and p16INK4a retain the capacity to undergo senescence via HIRA/PML pathway through suppressing the canonical Wnt signaling. Although the levels of total phosphorylated pRB were not decreased by Wnt5a, a decrease in the levels of pRBpS780 that is mediated by cyclin D1/CDK4 was observed (FIGS. 5C and D).

The data show Wnt5a promoter hypermethylation in a number of human EOC cell lines in which Wnt5a is down regulated (FIG. 2). The data also show that Wnt5a down regulation is an independent predictor for overall survival in EOC patients. The data showed that there is a difference in Wnt5a expression between type I and type II EOC (P ¼ 0.005; Table 1).

In summary, these data show that Wnt5a is often expressed at lower levels in human EOCs compared with either normal human ovarian surface epithelium or fallopian tube epithelium. A lower level of Wnt5a expression correlates with tumor stage and predicts shorter overall survival in EOC patients. Reconstitution of Wnt5a signaling inhibits the growth of human EOC cells both in vitro and in vivo. In addition, Wnt5a reconstitution suppresses the proliferation-promoting canonical Wnt/β-catenin signaling in human EOC cells. Significantly, Wnt5a reconstitution drives cellular senescence in human EOC cells and this correlates with activation of the senescence-promoting HIRA/PML pathway.

The invention is not limited to the embodiments described and exemplified above, but is capable of variation and modification within the scope of the appended claims.

We claim:

1. A method, comprising detecting Wnt5a protein levels that are lower than the Wnt5a protein levels of normal ovarian epithelial cells or normal fallopian tube epithelial cells in an epithelial ovarian tumor, and inducing senescence in cells of the tumor by contacting the cells with an amount of Wnt5a protein, or biologically active fragment thereof, effective to activate the histone repressor A (HIRA)/promyelocytic leukemia (PML) pathway in the cells, thereby inducing senescence in the cells of the tumor.

2. The method of claim 1, wherein the biologically active fragment of the Wnt5a protein is the N-terminally formylated peptide comprising SEQ ID NO: 2.

3. The method of claim 1, further comprising quantifying the level of Wnt5a protein detected in the epithelial ovarian tumor.

4. A method, comprising detecting Wnt5a mRNA levels that are lower than the Wnt5a mRNA levels of normal ovarian epithelial cells or normal fallopian tube epithelial cells in an epithelial ovarian tumor, and inducing senescence in cells of the tumor by contacting the cells with an amount of Wnt5a protein, or biologically active fragment thereof, effective to activate the histone repressor A (HIRA)/promyelocytic leukemia (PML) pathway in the cells, thereby inducing senescence in the cells of the tumor.

5. The method of claim 4, wherein the biologically active fragment of the Wnt5a protein is the N-terminally formylated peptide comprising SEQ ID NO: 2.


SEQUENCE LISTING (2 sequences)

SEQ ID NO: 1 - DNA, 5855 bp, Homo sapiens
agttgcctgcgcgccctcgccggaccggcggctccctagttgcgccccgaccaggccctgcccttgctgccggctcgcgcgcgtccgcgccccctccattcctgggcgcatcccagctctgccccaactcgggagtccaggcccgggcgccagtgcccgcttcagctccggttcactgcgcccgccggacgcgcgccggaggactccgcagccctgctcctgaccgtccccccaggcttaacccggtcgctccgctcggattcctcggctgcgctcgctcgggtggcgacttcctccccgcgccccctccccctcgccatgaagaagtccattggaatattaagcccaggagttgctttggggatggctggaagtgcaatgtcttccaagttcttcctagtggctttggccatatttttctccttcgcccaggttgtaattgaagccaattcttggtggtcgctaggtatgaataaccctgttcagatgtcagaagtatatattataggagcacagcctctctgcagccaactggcaggactttctcaaggacagaagaaactgtgccacttgtatcaggaccacatgcagtacatcggagaaggcgcgaagacaggcatcaaagaatgccagtatcaattccgacatcgaaggtggaactgcagcactgtggataacacctctgtttttggcagggtgatgcagataggcagccgcgagacggccttcacatacgcggtgagcgcagcaggggtggtgaacgccatgagccgggcgtgccgcgagggcgagctgtccacctgcggctgcagccgcgccgcgcgccccaaggacctgccgcgggactggctctggggcggctgcggcgacaacatcgactatggctaccgctttgccaaggagttcgtggacgcccgcgagcgggagcgcatccacgccaagggctcctacgagagtgctcgcatcctcatgaacctgcacaacaacgaggccggccgcaggacggtgtacaacctggctgatgtggcctgcaagtgccatggggtgtccggctcatgtagcctgaagacatgctggctgcagctggcagacttccgcaaggtgggtgatgccctgaaggagaagtacgacagcgcggcggccatgcggctcaacagccggggcaagttggtacaggtcaacagccgcttcaactcgcccaccacacaagacctggtctacatcgaccccagccctgactactgcgtgcgcaatgagagcaccggctcgctgggcacgcagggccgcctgtgcaacaagacgtcggagggcatggatggctgcgagctcatgtgctgcggccgtggctacgaccagttcaagaccgtgcagacggagcgctgccactgcaagttccactggtgctgctacgtcaagtgcaagaagtgcacggagatcgtggaccagtttgtgtgcaagtagtgggtgccacccagcactcagccccgctcccaggacccgcttatttatagaaagtacagtgattctggtttttggtttttagaaatattttttatttttccccaagaattgcaaccggaaccattttttttcctgttaccatctaagaactctgtggtttattattaatattataattattatttggcaataatgggggtgggaaccaagaaaaatatttattttgtggatctttgaaaaggtaatacaagacttcttttgatagtatagaatgaaggggaaataacacataccctaacttagctgtgtggacatggtacacatccagaaggtaaagaaatacattttctttttctcaaatatgccatcatatgggatgggtaggttccagttgaaagagggtggtagaaatctattcacaattcagcttctatgaccaaaatgagttgtaaattctctggtgcaagataaaaggtcttgggaaaacaaaacaaaacaaaacaaacctcccttccccagcagggctgctagcttgctttctgcattttcaaaatgataatttacaatggaaggacaagaatgtcatattctcaaggaaaaaaggtatatcacatgtctcattctcctcaaatattccatttgcagacagaccgtcatattctaatagctcatgaaatttgggcagcagggaggaaagtccccagaaattaaaaaatttaaaactcttatgtcaagatgttgatttgaagctgttataagaattaggattccagattgtaaaaagatccccaaatgattctggacactagatttttttgtttggggaggttggcttgaacataaatgaaaatatcctgttattttcttagggatacttggttagtaaattataatagtaaaaataatacatgaatcccattcacaggttctcagcccaagcaacaaggtaattgcgtgccattcagcactgcaccagagcagacaacctatttgaggaaaaacagtgaaatccaccttcctcttcacactgagccctctctgattcctccgtgttgtgatgtgatgctggccacgtttccaaacggcagctccactgggtcccctttggttgtaggacaggaaatgaaacattaggagctctgcttggaaaacagttcactacttagggatttttgtttcctaaaacttttattttgaggagcagtagttttctatgttttaatgacagaacttggctaatggaattcacagaggtgttgcagcgtatcactgttatgatcctgtgtttagattatccactcatgcttctcctattgtactgcaggtgtaccttaaaactgttcccagtgtacttgaacagttgcatttataaggggggaaatgtggtttaatggtgcctgatatctcaaagtcttttgtacataacatatatatatatatacatatatataaatataaatataaatatatctcattgcagccagtgatttagatttacagtttactctggggttatttctctgtctagagcattgttgtccttcactgcagtccagttgggattattccaaaagttttttgagtcttgagcttgggctgtggccctgctgtgatcataccttgagcacgacgaagcaaccttgtttctgaggaagcttgagttctgactcactgaaatgcgtgttgggttgaagatatcttttttcttttctgcctcacccctttgtctccaacctccatttctgttcactttgtggagagggcattacttgttcgttatagacatggacgttaagagatattcaaaactcagaagcatcagcaatgtttctcttttcttagttcattctgcagaatggaaacccatgcctattagaaatgacagtacttattaattgagtccctaaggaatattcagcccactacatagatagcttttttttttttttttttaataaggacacctctttccaaacagtgccatcaaatatgttcttatctcagacttacgttgttttaaaagtttggaaagatacacatctttcataccccccttaggcaggttggctttcatatcacctcagccaactgtggctcttaatttattgcataatgatattcacatcccctcagttgcagtgaattgtgagcaaaagatcttgaaagcaaaaagcactaattagtttaaaatgtcacttttttggtttttattatacaaaaaccatgaagtactttttttatttgctaaatcagattgttcctttttagtgactcatgtttatgaagagagttgagtttaacaatcctagcttttaaaagaaactatttaatgtaaaatattctacatgtcattcagatattatgtatatcttctagcctttattctgtacttttaatgtacatatttctgtcttgcgtgatttgtatatttcactggtttaaaaaacaaacatcgaaaggcttatgccaaatggaagatagaatataaaataaaacgttacttgtatattggtaagtggtttcaattgtccttcagataattcatgtggagatttttggagaaaccatgacggatagtttaggatgactacatgtcaaagtaataaaagagtggtgaattttaccaaaaccaagctatttggaagcttcaaaaggtttctatatgtaatggaacaaaaggggaattctcttttcctatatatgttccttacaaaaaaaaaaaaaaaagaaatcaagcagatggcttaaagctggttataggattgctcacattcttttagcattatgcatgtaacttaattgttttagagcgtgttgctgttgtaacatcccagagaagaatgaaaaggcacatgcttttatccgtgaccagatttttagtccaaaaaaatgtatttttttgtgtgtttaccactgcaactattgcacctctctatttgaatttactgtggaccatgtgtggtgtctctatgccctttgaaagcagtttttataaaaagaaagcccgggtctgcagagaatgaaaactggttggaaactaaaggttcattgtgttaagtgcaattaatacaagttattgtgcttttcaaaaatgtacacggaaatctggacagtgctgcacagattgatacattagcctttgctttttctctttccggataaccttgtaacatattgaaaccttttaaggatgccaagaatgcattattccacaaaaaaacagcagaccaacatatagagtgtttaaaatagcatttctgggcaaattcaaactcttgtggttctaggactcacatctgtttcagtttttcctcagttgtatattgaccagtgttctttattgcaaaaacatatacccgatttagcagtgtcagcgtattttttcttctcatcctggagcgtattcaagatcttcccaatacaagaaaattaataaaaaatttatatataggcagcagcaaaagagccatgttcaaaatagtcattatgggctcaaatagaaagaagacttttaagttttaatccagtttatctgttgagttctgtgagctactgacctcctgagactggcactgtgtaagttttagttgcctaccctagctcttttctcgtacaattttgccaataccaagtttcaatttgtttttacaaaacattattcaagccactagaattatcaaatatgacgctatagcagagtaaatactctgaataagagaccggtactagctaactccaagagatcgttagcagcatcagtccacaaacacttagtggcccacaatatatagagagatagaaaaggtagttataacttgaagcatgtatttaatgcaaataggcacgaaggcacaggtctaaaatactacattgtcactgtaagctatacttttaaaatatttattttttttaaagtattttctagtcttttctctctctgtggaatggtgaaagagagatgccgtgttttgaaagtaagatgatgaaatgaatttttaattcaagaaacattcagaaacataggaattaaaacttagagaaatgatctaatttccctgttcacacaaactttacactttaatctgatgattggatattttattttagtgaaacatcatcttgttagctaactttaaaaaatggatgtagaatgattaaaggttggtatgatttttttttaatgtatcagtttgaacctagaatattgaattaaaatgctgtctcagtattttaaaagcaaaaaaggaatggaggaaaattgcatcttagaccatttttatatgcagtgtacaatttgctgggctagaaatgagataaagattatttatttttgttcatatcttgtacttttctattaaaatcattttatgaaatccaaaaaaaaaaaaaaaaaa

SEQ ID NO: 2 - protein, 6 aa, Homo sapiens
  MOD_RES (1)..(1): FORMYLATION (M at position 1 may optionally be formylated)
MDGCEL